(19) United States
(12) Patent Application Publication
(10) Pub. No.: US 2024/0327784 A1
(43) Pub. Date: Oct. 3, 2024

(54) METHOD OF PRODUCING CULTURE SOLUTION FOR HUMAN OR ANIMAL CELL CULTURE

(71) Applicants: DAIWA CAN COMPANY, Tokyo (JP); IntegriCulture Inc., Bunkyo-ku (JP)

(72) Inventors: Mami Horiuchi, Sagamihara-shi (JP); Toshiyuki Akachi, Sagamihara-shi (JP); Takashi Kawada, Sagamihara-shi (JP); Ryoji Fujii, Sagamihara-shi (JP); Ikko Kawashima, Bunkyo-ku (JP); Keita Tanaka, Bunkyo-ku (JP)

(73) Assignees: DAIWA CAN COMPANY, Tokyo (JP); IntegriCulture Inc., Bunkyo-ku (JP)

(21) Appl. No.: 18/741,314

(22) Filed: Jun. 12, 2024

Related U.S. Application Data
(63) Continuation of application No. PCT/JP2022/045500, filed on Dec. 9, 2022.

(30) Foreign Application Priority Data
Dec. 13, 2021 (JP) 2021-201542

(51) Int. Cl. C12N 5/00

(52) U.S. Cl. CPC C12N 5/0018; C12N 2500/24

(57) ABSTRACT

A method of producing a culture solution for human or animal cell culture includes bringing an iron supply source including an iron-containing section and a plating layer provided thereon into contact with an aqueous medium to supply iron from the iron supply source to the aqueous medium.

### CROSS-REFERENCE TO RELATED APPLICATION

This application is a Continuation Application of PCT Application No. PCT/JP2022/045500, filed Dec. 9, 2022 and based upon and claiming the benefit of priority from Japanese Patent Application No. 2021-201542, filed Dec. 13, 2021, the entire contents of all of which are incorporated herein by reference.

### FIELD

The present invention relates to technology to culture human or animal cells.

### BACKGROUND

Culture of human or animal cells is technology essential to regenerative medicine and production of edible meat. The culture of human or animal cells may also be used to produce other products, such as ingredients for use in cosmetics or food such as supplements.

For example, humans have several grams of iron in their bodies as an essential element for life support. It is thus desirable that the culture solution for culturing cells contain iron as well as in vivo environment. If, however, iron is added to the culture solution, radicals are generated during the oxidation of iron to trivalent iron ions through divalent iron ions and the reduction of trivalent iron ions to divalent iron ions. Therefore, environment in excess of iron ions is detrimental to the cells (see Patent Literature 1).

Note that the addition of iron to a culture medium may be carried out in the culture of Euglena or the like (see Patent Document 2).

### CITATION LIST

PATENT LITERATURE

| | |
|---|---|
| Patent | Jpn. Pat. Appln. KOKAI Publication No. 2020-54764 |
| Literature 1 | |
| Patent | Jpn. Pat. Appln. KOKAI Publication No. 2016-195579 |
| Literature 2 | |

### SUMMARY

Living bodies have a function of controlling the amount and form of iron therein. In contrast, a cell culture environment generally does not have such a control function. As described above, the environment in excess of iron ions is detrimental to the cells.

It is therefore an object of the present invention to provide a technology that facilitates the control of iron concentration in a culture solution for use in human or animal cell culture.

According to an aspect of the present invention, there is provided a method of producing a culture solution for human or animal cell culture, comprising bringing an iron supply source including an iron-containing section and a plating layer provided thereon into contact with an aqueous medium to supply iron from the iron supply source to the aqueous medium.

According to another aspect of the present invention, there is provided the method of producing the culture solution for human or animal cell culture according to the above aspect, wherein the iron supply source has a plate shape.

According to still another aspect of the present invention, there is provided the method of producing the culture solution for human or animal cell culture according to the above aspect, wherein the iron-containing section is exposed at an end face of the iron supply source.

According to still another aspect of the present invention, there is provided the method of producing the culture solution for human or animal cell culture according to any one of the above aspects, wherein the iron-containing section is made of steel.

According to still another aspect of the present invention, there is provided the method of producing the culture solution for human or animal cell culture according to any one of the above aspects, wherein the plating layer contains nickel or chromium.

According to still another aspect of the present invention, there is provided the method of producing the culture solution for human or animal cell culture according to any one of the above aspects, further comprising: determining a second target value of iron concentration in the aqueous medium after iron is supplied by the iron supply source based on a first target value of iron concentration in the culture solution for human or animal cell culture; and determining a condition for supply of iron from the iron supply source to the aqueous medium that allows the iron concentration in the aqueous medium to be equal to the second target value, wherein the iron is supplied from the iron supply source to the aqueous medium under the condition for supply.

According to still another aspect of the present invention, there is provided the method of producing the culture solution for human or animal cell culture according to the above aspect, wherein the determining the condition for supply comprises selecting, as the iron supply source, one whose plating layer contains a metal having a higher ionization tendency than that of iron if the second target value is lower than a reference value, and selecting, as the iron supply source, one whose plating layer contains a metal having a lower ionization tendency than that of iron if the second target value is higher than the reference value.

According to still another aspect of the present invention, there is provided a culture solution for human or animal cell culture obtainable by the method of any one of the above aspects.

According to still another aspect of the present invention, there is provided a culture method comprising: producing the culture solution for human or animal cell culture by the method of any one of the above aspects; and culturing human or animal cells in the culture solution for human or animal cell culture.

According to still another aspect of the present invention, there is provided a culture production method comprising: producing the culture solution for human or animal cell culture by the method of any one of the above aspects; and culturing human or animal cells in the culture solution for human or animal cell culture.

According to still another aspect of the present invention, there is provided a culture obtainable by the culture production method of the above aspect.

According to still another aspect of the present invention, there is provided a human or animal cell secretion-containing solution production method, comprising: producing the culture solution for human or animal cell culture by the method of any one of the above aspects; culturing human or animal cells in the culture solution for human or animal cell culture; and separating from the cultured human or animal cells a mixed solution containing a component secreted by the human or animal cells during the culturing of the human or animal cells and the culture solution for human or animal cell culture.

According to still another aspect of the present invention, there is provided a human or animal cell secretion-containing solution obtainable by the human or animal cell secretion-containing solution production method of the above aspect.

According to still another aspect of the present invention, there is provided an iron supply source for use in producing a culture solution for human or animal cell culture, comprising supplying iron to an aqueous medium in contact with the aqueous medium, the iron supply source including an iron-containing section and a plating layer provided thereon.

According to still another aspect of the present invention, there is provided a culture solution production kit for human or animal cell culture, comprising the iron supply source of the above aspect, an aqueous medium, and a container.

According to the present invention, a technology that facilitates the control of iron concentration in a culture solution for use in human or animal cell culture is provided.

### BRIEF DESCRIPTION OF THE DRAWINGS

FIG. 1 is a perspective view schematically showing an example of an iron supply source for use in a method of producing a culture solution for human or animal cell culture according to an embodiment of the present invention.

FIG. 2 is a graph showing an example of the effect of a plating layer on the supply of iron from an iron supply source to an aqueous medium.

FIG. 3 is a graph showing another example of the effect of the plating layer on the supply of iron from the iron source to the aqueous medium.

FIG. 4 is a graph showing an example of the change in iron concentration in the culture solution according to a culture period when the plating layer is omitted.

FIG. 5 is a graph showing an example of the change in iron concentration in the culture solution according to a culture period when a nickel-plated layer is provided as the plating layer.

FIG. 6 is a graph showing an example of the change in iron concentration in the culture solution according to a culture period when a chromium-plated layer is provided as the plating layer.

### DETAILED DESCRIPTION

An embodiment of the present invention will be described below with reference to the drawings. It should be noted that the matters to be described below can be incorporated alone or in combination into the method or product according to any of the foregoing aspects.

A method of producing a culture solution for human or animal cell culture according to an embodiment of the present invention includes supplying iron from an iron supply source to an aqueous medium by bringing the iron supply source into contact with the aqueous medium.

FIG. 1 is a perspective view schematically showing an example of an iron supply source for use in the method of producing a culture solution for human or animal cell culture according to the embodiment of the present invention.

The iron supply source 1 shown in FIG. 1 has a plate shape. Here, the iron supply source 1 has the shape of a flat plate, and the shape of an orthogonal projection onto a plane perpendicular to the thickness direction (hereinafter referred to as a planar shape) is square. The planar shape of an iron-containing section 11 may be a circle, an ellipse, an oval, a triangle, and other shapes such as a polygon having five or more corners.

Although the iron supply source 1 has no through hole, it may have one or more through holes. With a through hole, the total length of the outline of the orthogonal projection is greater than in the case of no through holes. If the total length is increased, iron is easily eluted from the iron supply source 1 into the aqueous medium.

The iron supply source 1 need not have the shape of a flat plate. The iron supply source 1 may be formed in a shape other than the flat plate shape, such as a corrugated plate shape. This structure is advantageous in decreasing the area of contact between the inner surface of a container containing the aqueous medium and the iron supply source 1 when the iron supply source 1 is placed in the container. The structure is also advantageous in decreasing the area of contact between a plurality of iron supply sources 1 that are placed in a container containing the aqueous medium. If the area of contact is decreased, the variation of the amount of iron eluted into the aqueous medium is reduced.

The iron supply source 1 need not have the shape of a plate. However, the iron supply source 1 having no plate shape makes it more difficult to control the area of an exposed portion of the iron-containing section 11 in the iron supply source 1 than in the case where the iron supply source 1 has the shape of a plate.

The dimensions of the iron supply source 1 can be set appropriately according to, for example, the volume of a container containing the iron supply source 1 and the aqueous medium, the volume of the aqueous medium supplied to the container, the number of iron supply sources 1 placed in the container and the structure and composition of the iron supply source 1. If the iron supply source 1 has a plate shape, the area of one of its main surfaces is in the range of 1 cm² to 100 cm² as an example and in the range of 8100 cm² to 12100 cm² as another example. In addition, the total length of the outline of the orthographic projection is in the range of 4 cm to 20 cm as an example and in the range of 360 cm to 440 cm as another example.

The iron supply source 1 includes the iron-containing section 11 and plating layers 12a and 12b provided thereon.

The iron-containing section 11 is a base material containing iron.

The iron-containing section 11 may be made of iron or may be made of iron and one or more other elements. In the latter case, the iron-containing section 11 preferably contains iron and one or more other metals, and more preferably, an iron alloy. The iron-containing section 11 is preferably made of steel.

The iron-containing section 11 has substantially the same shape as that of the iron supply source 1. That is, the iron-containing section 11 has a flat plate shape and a square planar shape. If the planar shape of the iron-containing section 11 is changed with the constant area of an orthogonal projection onto a plane perpendicular to the thickness direction of the iron-containing section 11, the circumference of the iron-containing section 11 is changed. If the circumference is increased, iron is easily eluted from the iron supply source 1 into the aqueous medium.

Although the iron-containing section 11 has no through hole, it may have one or more through holes as described above with respect to the iron supply source 1. With a through hole, the total length of the outline of the orthogonal projection is greater than in the case of no through holes. If the total length is increased, iron is easily eluted from the iron supply source 1 into the aqueous medium.

The easiness of iron elution from the iron supply source 1 into the aqueous medium can be changed by changing the thickness of the iron-containing section 11. For example, if the iron-containing section 11 is thickened, iron is easily eluted from the iron supply source 1 into the aqueous medium.

As described above with respect to the iron supply source 1, the iron-containing section 11 need not have the shape of a flat plate. The iron-containing section 11 may be formed in a shape other than the flat plate, such as a corrugated plate shape. The iron-containing section 11 need not be shaped like a plate.

If the iron-containing section 11 has a plate shape, its thickness is in the range of 0.1 mm to 0.25 mm as an example and in the range of 0.1 mm to 10 mm as another example.

The plating layers 12a and 12b cover their respective main surfaces of the iron-containing section 11. Neither of the plating layers 12a and 12b cover the end face of the iron-containing section 11. That is, the plating layers 12a and 12b cause the iron-containing section 11 to be exposed at the end face of the iron supply source 1.

At least one of the plating layers 12a and 12b may further cover the iron-containing section 11 at the end face of the iron supply source 1. If, however, the plating layers 12a and 12b cause the iron-containing section 11 to be exposed at the end face of the iron supply source 1, iron is more easily eluted from the iron supply source 1 into the aqueous medium than in the case where at least one of the plating layers 12a and 12b further covers the iron-containing section 11 at the end face of the iron supply source 1.

The plating layers 12a and 12b suppress the elution of iron from the iron-containing section 11 into the aqueous medium. This effect occurs at least in part because the plating layers 12a and 12b decrease the area of contact between the iron-containing section 11 and the aqueous medium. If at least one of the plating layers 12a and 12b contains a metal whose ionization tendency is higher than that of iron, part of the effect is brought about by the metal-containing plating layer acting as a sacrificial layer. That is, if at least one of the plating layers 12a and 12b contains a metal whose ionization tendency is higher than that of iron, the metal is more easily ionized in the aqueous medium than iron and thus suppresses the elution of iron.

The plating layers 12a and 12b are made of metals other than iron. The plating layers 12a and 12b may be made of a metal whose ionization tendency is higher than that of iron or a metal whose ionization tendency is lower than that of iron. Metals whose ionization tendency is higher than that of iron are, for example, chromium and zinc.

Metals whose ionization tendency is lower than that of iron are, for example, nickel and tin. As one example, the plating layers 12a and 12b contain nickel or chromium.

Each of the plating layers 12a and 12b may contain only one metal or may contain two or more metals. The type of metal contained in the plating layer 12a and the type of metal contained in the plating layer 12b may be the same or different.

If the plating layer 12a is formed of a metal whose ionization tendency is higher than that of iron, the plating layer 12b may be made of a metal whose ionization tendency is higher than that of iron or a metal whose ionization tendency is lower than that of iron. If the plating layer 12a is made of a metal whose ionization tendency is lower than that of iron, the plating layer 12b may be made of a metal whose ionization tendency is higher than that of iron or a metal whose ionization tendency is lower than that of iron.

Each of the plating layers 12a and 12b may have a single-layer structure or a multilayer structure. Note that if each of the plating layers 12a and 12b has a multilayer structure including a layer made of a metal whose ionization tendency is higher than that of iron and a most superficial layer made of a metal whose ionization tendency is lower than that of iron, the former layer does not act as a sacrificial layer because the area of contact with the aqueous medium is small.

Note that the iron supply source 1 can be distributed alone. Alternatively, the iron supply source 1 can be distributed in the form of a culture solution production kit for human or animal cell culture including the iron supply source, an aqueous medium and a container containing the aqueous medium.

The aqueous medium is, for example, supplied with iron from the iron supply source 1 and used as a culture solution for human or animal cell culture. Alternatively, the aqueous medium is supplied with iron from the iron supply source 1 and further mixed with one or more other components or solutions into a mixture. This mixture is used as a culture solution for human or animal cell culture.

As the aqueous medium, for example, a solution that differs from the final culture solution for human or animal cell culture only in that it contains no eluted material from the iron supply source 1 can be used. Alternatively, as the aqueous medium, a solution that differs from the final culture solution for human or animal cell culture only in that it contains neither eluted material from the iron supply source 1 nor one or more other components can be used. The other one or more components are, for example, inorganic salt; amino acid; vitamin; sugar; and one or more bioactive proteins that contribute to signal transduction between cells, such as growth factors, hormones and cytokines. The aqueous medium may be water, preferably a solution containing inorganic salt, and more preferably a solution containing inorganic salt, amino acid and vitamin.

The concentration of inorganic salt in the aqueous medium is preferably within the range of 0 mg/L to 20000 mg/L and more preferably within the range of 600 mg/L to 11000 mg/L. The inorganic salt may promote the supply of iron from the iron supply source 1 to the aqueous medium.

The aqueous medium can be brought into contact with the iron supply source 1 in a variety of ways.

For example, the iron supply source 1 may be immersed in an aqueous medium contained in a container. In this case, the aqueous medium may be stirred or circulated in a circulation path including the container. When an oxide film is formed on the exposed surface of the iron-containing section 11, at least part of the oxide film can be removed therefrom by the flow of the aqueous medium.

Alternatively, the aqueous medium may be discharged toward the iron supply source 1. In this case, the aqueous medium may be circulated in a circulation path including a nozzle head having a nozzle for discharging the aqueous medium and a container for recovering the aqueous medium discharged to the iron supply source 1.

If the aqueous medium is brought into contact with the iron supply source 1, iron is eluted from the iron-containing section 11 into the aqueous medium. The aqueous medium into which an appropriate amount of iron is eluted can, for example, itself be used as a culture solution for human or animal cell culture. Alternatively, the aqueous medium into which an appropriate amount of iron is eluted is mixed with one or more other solutions. In this case, the resulting mixture can be used as a culture solution for human or animal cell culture.

The rate of increase of the iron concentration in the aqueous medium varies according to, for example, the composition of the aqueous medium and the structure and composition of the iron supply source 1. Thus, the conditions for supply of iron from the iron supply source 1 to an aqueous medium to be used need to be determined appropriately in accordance with the composition of the aqueous medium. The conditions for supply of iron from the iron supply source 1 to the aqueous medium are determined, for example, by the following method.

First, a first target value of the iron concentration in a culture solution for human or animal cell culture is set. For example, a human or animal cell is cultured using culture solutions having different iron concentrations. The most desirable iron concentration is obtained from the result and is set as the first target value.

Next, a second target value of the iron concentration in the aqueous medium after iron supply from the iron supply source 1 is determined based on the first target value. If the aqueous medium after the iron supply from the iron supply source 1 is used as a culture solution for human or animal cell culture, the second target value is, for example, approximately equal to the first target value. If the aqueous medium after the iron supply from the iron supply source 1 is mixed with one or more other solutions and the resulting mixed solution is used as a culture solution for human or animal cell culture, the iron concentration in the aqueous medium after the iron supply, which achieves the first target value in the culture solution, is calculated, for example, from the mixing ratio of the solutions and the first target value, and a value approximately equal to the value obtained from the calculation is set as the second target value.

Then, the conditions for supply of iron from the iron supply source 1 to the aqueous medium is determined such that the iron concentration in the aqueous medium is allowed to be equal to the second target value.

For example, if the second target value is lower than a reference value, as the iron supply source 1, an iron supply source is selected in which the plating layers 12a and 12b contain metal whose ionization tendency is higher than that of iron. If the second target value is higher than the reference value, as the iron supply source 1, an iron supply source is selected in which the plating layers 12a and 12b contain metal whose ionization tendency is lower than that of iron. The reference value here is, for example, a value that is larger than the iron concentration achieved in the case of using the iron supply source 1 whose plating layers 12a and 12b contain metal having an ionization tendency higher than that of iron and that is smaller than the iron concentration achieved in the case of using the iron supply source 1 whose plating layers 12a and 12b contain metal having an ionization tendency lower than that of iron.

As described above, if the total length of the outline of the orthogonal projection to the plane perpendicular to the thickness direction of the iron supply source 1 is increased, iron is easily eluted from the iron supply source 1 into the aqueous medium. In addition, if the number of iron supply sources 1 that are in contact with the aqueous medium is increased, the iron concentration in the aqueous medium becomes high. Therefore, for example, after the selection described above, the total length and the number of iron supply sources 1 may be set so that the iron concentration in the aqueous medium is equal to the second target value.

After that, iron is supplied from the iron supply source 1 to the aqueous medium under the supply conditions determined as described above.

The culture solution for human or animal cell culture is produced as described above.

The above method facilitates the control of the iron concentration in the culture solution for use in culturing human or animal cells. This will be described below.

If the plating layers 12a and 12b are omitted, the iron concentration in the aqueous medium increases greatly with an increase in the contact time. The desired iron concentration in the final culture solution for human or animal cell culture varies depending on the type of human or animal cells to be cultured, but is low in any case. If, therefore, the iron concentration in the aqueous medium increases greatly with an increase in the contact time, it is necessary to, for example, measure the iron concentration each time the aqueous medium supplied with iron is used as a culture solution for human or animal cell culture or a part thereof.

In contrast, since the iron supply source 1 includes the plating layers 12a and 12b, the iron concentration in the aqueous medium moderately increases with an increase in the contact time. If, therefore, the iron supply source 1 is used, the range of the contact time in which the iron concentration is allowable is wide. In this case, it is easy to control the iron concentration in the culture solution for use in culturing human or animal cells.

The culture solution produced by the above method can be used as that for human or animal cell culture. The cells to be cultured using the culture solution for human or animal cell culture may be any cells if they are human or animal cells. The human or animal cells may be undifferentiated cells or differentiated cells. The human or animal cells may constitute tissues or organs, or alternatively may not constitute tissues or organs.

The culture of human or animal cells using the culture solution for human or animal cell culture can be carried out by a variety of methods. The culture of human or animal cells may be, for example, suspension culture or adhesion culture. This culture may be performed in the presence or absence of scaffolding. In addition, the culture may involve cell division, differentiation, fusion, and the like, or may not involve them.

Cultures obtained by culturing human or animal cells can be used for regenerative medicine, for example. The cultures may be other articles such as edible meat.

Human or animal cells secrete various components as they are cultured. In using the components, for example, a mixed solution containing a component secreted by the human or animal cells during the human or animal cell culture and a culture solution for human or animal cell culture is separated from the cultured human or animal cells. The mixed solution, that is, a human or animal cell secretion-containing solution can be used, for example, as a culture solution for culturing other human or animal cells or a part thereof. Alternatively, the human or animal cell secretion-containing solution or an extract, which is obtained by extracting one or more components from the solution and purifying the extract if necessary, can be used for the production of raw materials for use in food such as supplements or cosmetics.

### Example

The results of tests performed by the present inventors will be described below.

### <Test 1>

Difference in the amount of iron that is eluted into the aqueous medium in accordance with the structure and composition of the iron supply source was studied by the following method.

The following five different specimens A to E were prepared as iron supply sources.

### Specimen A:

Steel plate material having no plating layer (which may be referred to as no plating hereinafter)

### Specimen B:

Steel plate material having a nickel-plated layer on both sides (which may be referred to as a Ni-plated steel plate)

### Specimen C:

Steel plate material having on both sides a plating layer of a multilayer structure in which a nickel layer, a tin layer and a chromium layer are formed in this order (which may be referred to as an LTS material)

### Specimen D:

Steel plate material having a tin-plated layer on both side (which may be referred to as a tin material)

### Specimen E:

Steel plate material having a chrome-plated layer on both sides (which may be referred to as a TFS material.)

The steels of the specimens A to E have the same composition. The thickness of the specimen A and the thickness of the iron-containing section of each of the specimens B to E are the same. Each of the specimens A to E has a square shape whose side is 3 cm in orthogonal projection to a plane perpendicular to the thickness direction. Each of the specimens B to E is obtained by cutting out of a plate material having a plating layer on both sides, and an iron-containing section is exposed at each of the four end faces thereof.

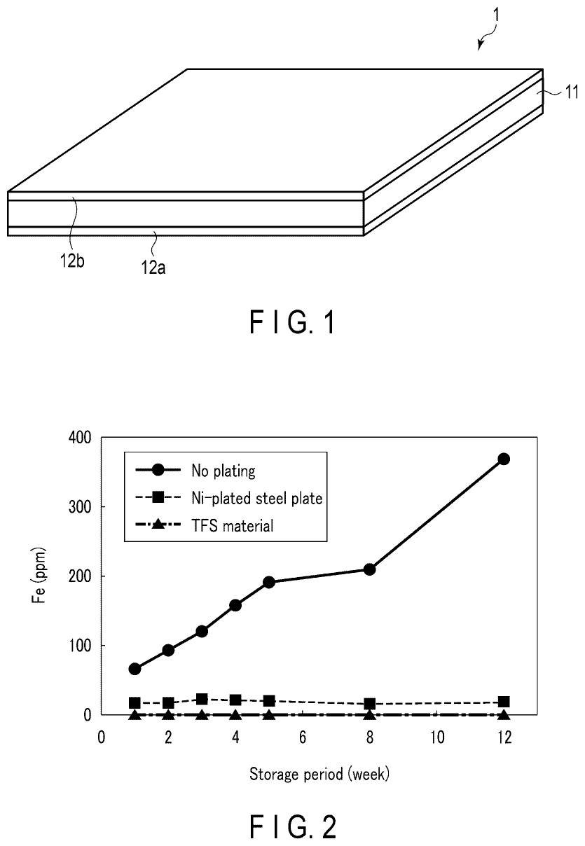

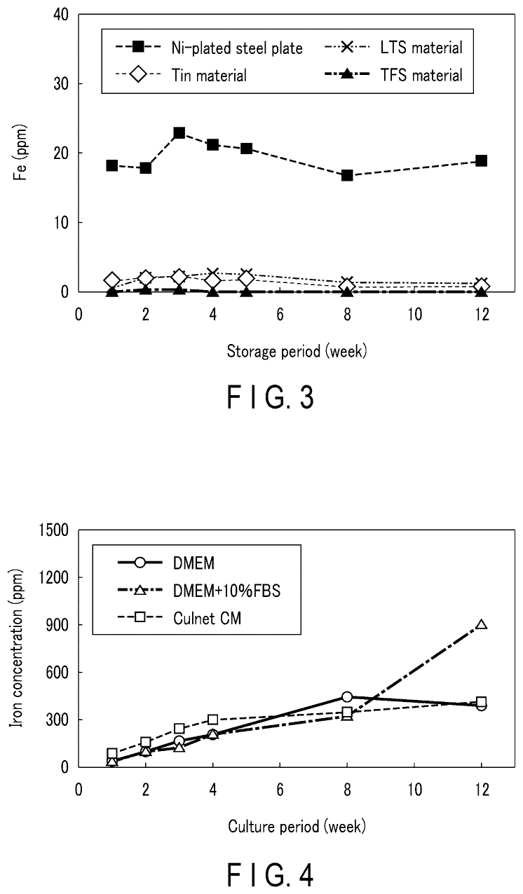

Next, 200 mL of aqueous medium was placed into a 250 mL heat-resistant glass bottle sterilized by autoclaving. Dulbecco's Modified Eagle Medium (DMEM) commercially available from FUJIFILM Wako Pure Chemical Corporation under code number D-43 30085 or D-41 30081 was used as the aqueous medium. The specimen A was washed with an aqueous ethanol solution containing ethanol at a concentration of 70 mass %, wiped off the surface solution, and then placed into the aqueous medium. The glass bottle containing the aqueous medium and specimen A was stored at 37° C., 10 mL of aqueous medium was sampled at a point of time of each of 1 week, 2 weeks, 3 weeks, 4 weeks, 8 weeks and 12 weeks after the specimen A was placed, and the concentration of iron in the aqueous medium was measured using an inductively coupled plasma (ICP) emission spectrophotometer. Tests and measurements were carried out in the same manner as above except that the specimens B to E were used instead of the specimen A. The results are shown in FIGS. 2 and 3.

FIG. 2 is a graph showing an example of the effect of a plating layer on the supply of iron from an iron supply source to an aqueous medium. FIG. 3 is a graph showing another example of the effect of the plating layer on the supply of iron from the iron supply source to the aqueous medium. In FIGS. 2 and 3, the term “storage period” represents the time elapsed after a specimen was placed into the aqueous medium.

As shown in FIG. 2, when the specimen A having no plating layer was used, the iron concentration in the aqueous medium increased greatly with an increase in the storage period. In contrast, as shown in FIGS. 2 and 3, when the specimens B to E were used, the iron concentration in the aqueous medium hardly increased with an increase in the storage period and was maintained at a much lower value than when the specimen A was used.

As shown in FIG. 3, the iron concentration in the aqueous medium was lower than when the specimens C to E were used and when the specimen B was used. That is, when a specimen containing a metal whose ionization tendency is higher than that of iron in its topmost layer was used, the iron concentration in the aqueous medium could be lower than when a specimen containing a metal whose ionization tendency is lower than that of iron in its topmost layer.

### <Test 2>

Difference in the amount of iron that is eluted into the aqueous medium in accordance with the structure and composition of the iron supply source and the composition of the aqueous medium was studied by the following method.

100 mL of aqueous medium was placed into a 250 mL heat-resistant glass bottle sterilized by autoclaving. The same DMEM as used in test 1 was used as the aqueous medium. The specimen A was washed with an aqueous ethanol solution containing ethanol at a concentration of 70 mass %, wiped off the surface solution, and then placed into the aqueous medium. The glass bottle containing the aqueous medium and specimen A was stored at 37° C. 10 mL of the aqueous medium was sampled at a point of time of each of 1 week, 2 weeks, 3 weeks, 4 weeks, 8 weeks and 12 weeks after the specimen A was placed, and the iron concentration in the aqueous medium was measured using the ICP optical emission spectrometer.

The same tests and measurements were performed as described above, except that a mixture of DMEM and fetal bovine serum (FBS) was used as the aqueous medium instead of DMEM. As the DMEM, the same DMEM as used in test 1 was used. The FBS concentration in the mixture was 10 mass % (hereinafter, this mixture may be referred to as “DMED+10% FBS”).

In addition, the same tests and measurements were carried out as described above, except that instead of using DMEM manufactured by FUJIFILM Wako Pure Chemical Corporation, a culture medium manufactured using the CulNet (registered trademark) System of IntegriCulture Inc. (which may be referred to as “CulNet CM” hereinafter) was used as the aqueous medium. Note that the CulNet (registered trademark) System is a culture solution production system that simulates an in vivo environment and produces substances secreted by organs using culture technology to enable the production of a culture medium containing the above substances without using serum.

The same tests and measurements were carried out as above except that the specimens B and E were used instead of the specimen A.

The results are shown in FIGS. 4 to 6.

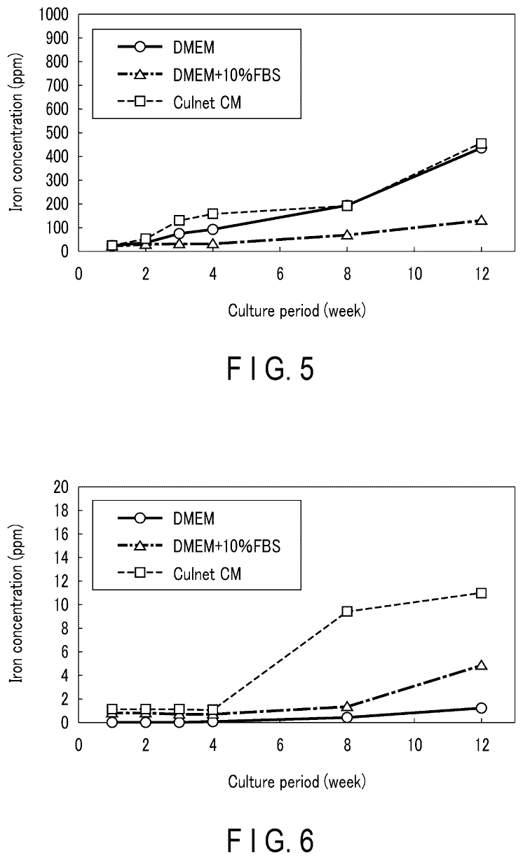

FIG. 4 is a graph showing an example of the change in iron concentration in the culture solution according to a culture period when the plating layer is omitted. FIG. 5 is a graph showing an example of the change in iron concentration in the culture solution according to a culture period when a nickel-plated layer is provided as the plating layer. FIG. 6 is a graph showing an example of the change in the iron concentration in the culture solution according to a culture period when the chromium-plated layer is provided as the plating layer. In FIGS. 4 to 6, the term “culture period” represents the time elapsed after a specimen was placed into the aqueous medium. As shown in FIGS. 4 to 6, the composition of the aqueous medium did not greatly affect the iron concentration. As shown in FIGS. 5 and 6, the iron concentration in the aqueous medium can greatly be varied by changing the composition of the plating layer provided in the iron supply source.

When the specimen B was immersed in the aqueous medium, the iron concentration in the aqueous medium was 100 ppm to 500 ppm at a point of time of 12 weeks after the specimen B was placed, as shown in FIG. 5. This concentration is close to the iron concentration in human blood. On the other hand, when a mixture of DMEM and FBS was used as the aqueous medium, and the specimen E was immersed in the aqueous medium, the iron concentration in the aqueous medium was 3 ppm (0.05 mM) or less, which is considered to be preferable for culture using serum, until 8 weeks after the specimen E was placed, and was maintained at 5 ppm or less even after 12 weeks after the specimen E was placed, as shown in FIG. 6.

From the above, it was confirmed that when a culture solution having a composition similar to that of blood is produced, an iron supply source having, as the topmost surface layer, a layer formed of a metal whose ionization tendency is lower than that of iron was suitable. In addition, it was confirmed that when a culture solution having a composition similar to that of blood is produced, an iron supply source having, as the topmost surface layer, a layer formed of a metal whose ionization tendency is higher than that of iron was suitable.

In addition, the concentration of heavy metal in the aqueous medium was measured using an ICP emission spectrophotometer at a point of time after each of 8 weeks and 12 weeks after the specimen A is placed into a glass bottle containing the aqueous medium. As a result, arsenic, lead, cadmium, tin, copper and chromium (VI) were below the standard values specified in the Food Sanitation Act, regardless of the specimen and the aqueous medium which 10 were used.

### REFERENCE SIGNS LIST

1 . . . Iron supply source, 11 . . . Iron-containing section, 12a . . . Plating layer, 12b . . . Plating layer

What is claimed is:

1. A method of producing a culture solution for human or animal cell culture, comprising bringing an iron supply source including an iron-containing section and a plating layer provided thereon into contact with an aqueous medium to supply iron from the iron supply source to the aqueous medium.

2. The method of producing the culture solution for human or animal cell culture according to claim 1, wherein the iron supply source has a plate shape.

3. The method of producing the culture solution for human or animal cell culture according to claim 2, wherein the iron-containing section is exposed at an end face of the iron supply source.

4. The method of producing the culture solution for human or animal cell culture according to claim 1, wherein the iron-containing section is made of steel.

5. The method of producing the culture solution for human or animal cell culture according to claim 1, wherein the plating layer contains nickel or chromium.

6. The method of producing the culture solution for human or animal cell culture according to claim 1, further comprising:
determining a second target value of iron concentration in the aqueous medium after iron is supplied by the iron supply source based on a first target value of iron concentration in the culture solution for human or animal cell culture; and
determining a condition for supply of iron from the iron supply source to the aqueous medium that allows the iron concentration in the aqueous medium to be equal to the second target value,
wherein the iron is supplied from the iron supply source to the aqueous medium under the condition for supply.

7. The method of producing the culture solution for human or animal cell culture according to claim 6, wherein the determining the condition for supply comprises selecting, as the iron supply source, one whose plating layer contains a metal having a higher ionization tendency than that of iron if the second target value is lower than a reference value, and selecting, as the iron supply source, one whose plating layer contains a metal having a lower ionization tendency than that of iron if the second target value is higher than the reference value.

8. A culture solution for human or animal cell culture obtainable by the method of claim 1.

9. A culture method comprising:
producing the culture solution for human or animal cell culture by the method of claim 1; and
culturing human or animal cells in the culture solution for human or animal cell culture.

10. A culture production method comprising:
producing the culture solution for human or animal cell culture by the method of claim 1; and
culturing human or animal cells in the culture solution for human or animal cell culture.

11. A culture obtainable by the culture production method of claim 10.

12. A human or animal cell secretion-containing solution production method, comprising:
producing the culture solution for human or animal cell culture by the method of claim 1;
culturing human or animal cells in the culture solution for human or animal cell culture; and
separating from the cultured human or animal cells a mixed solution containing a component secreted by the human or animal cells during the culturing of the human or animal cells and the culture solution for human or animal cell culture.

13. A human or animal cell secretion-containing solution obtainable by the human or animal cell secretion-containing solution production method of claim 12.

14. An iron supply source for use in producing a culture solution for human or animal cell culture, comprising supplying iron to an aqueous medium in contact with the aqueous medium, the iron supply source including an iron-containing section and a plating layer provided thereon.

15. A culture solution production kit for human or animal cell culture, comprising the iron supply source of claim 14, an aqueous medium, and a container.
